(19) United States
(12) Patent Application Publication
(10) Pub. No.: US 2026/0124103 A1
(43) Pub. Date: May 7, 2026

(54) Adaptive Virtual Reality System for Sexual Wellness with Synchronized Haptic Feedback

(71) Applicant: Irina Turkina, Puerto Plata (DO)

(72) Inventor: Irina Turkina, Puerto Plata (DO)

(21) Appl. No.: 19/199,173

(22) Filed: May 5, 2025

(51) Int. Cl. A61H 19/00; A61M 21/00; A61M 21/02; G06F 3/01

(52) U.S. Cl. CPC A61H 19/00; A61M 21/02; G06F 3/013; A61H 2201/02; A61H 2201/1607; A61H 2201/5007; A61H 2201/5043; A61H 2201/5084; A61H 2230/065; A61H 2230/625; A61M 2021/0027; A61M 2021/005

(57) ABSTRACT

The present invention provides a virtual reality (VR) system that synchronizes smart stimulation devices (e.g., haptic actuators) with VR content in real time. A simulation engine dynamically adjusts tactile, thermal, or mechanical feedback based on user physiological responses (e.g., eye/hand tracking, heart rate). The system includes a VR headset with motion sensors, wirelessly connected smart devices, and adaptive software to personalize therapeutic experiences. Applications include sexual wellness and trauma recovery, where real-time feedback improves user safety and immersion. The architecture supports modular hardware/software expansion.

### 1. FIELD OF THE INVENTION

This invention relates to virtual reality (VR) systems, specifically a platform for delivering adaptive, sensor-driven intimate experiences. The system integrates a VR headset with biometric tracking (e.g., eye/hand motion, physiological signals) and wirelessly synchronized smart devices (e.g., haptic actuators) to provide real-time tactile, thermal, or mechanical feedback. A simulation engine dynamically adjusts VR content and device outputs based on user input, enabling personalized therapeutic applications in sexual wellness and trauma recovery.

### 2. BACKGROUND OF THE INVENTION

Sexual wellness is a fundamental aspect of human health, encompassing psychological, emotional, and physical dimensions. However, many individuals experience difficulty achieving satisfying or therapeutic intimacy due to a range of factors, including trauma, anxiety, body image concerns, mobility impairments, and the absence of emotionally safe environments for exploration. Traditional interventions—such as talk therapy or medical treatment—may provide partial support, but often lack experiential tools to rebuild confidence, physical comfort, or sexual agency.

Recent advances in virtual reality (VR) and smart devices have introduced new opportunities for interactive engagement. However, existing applications remain narrowly focused. VR platforms designed for adult content typically offer passive viewing experiences, with limited or unidirectional synchronization between content and devices. These systems do not account for emotional readiness, consent management, or adaptive pacing. Likewise, therapeutic VR platforms for pain, PTSD, or phobias focus on clinical desensitization and do not accommodate intimacy-related experiences.

Gaming-oriented VR further demonstrates the technical limitations of current systems for sexual wellness. Most platforms rely on handheld controllers, exaggerated gesture inputs, and goal-oriented scene design—elements which are ill-suited for emotionally sensitive or embodied experiences. Individuals seeking trauma recovery, physical reconnection, or fantasy exploration often find such interaction paradigms disruptive or alienating. Moreover, current interfaces rarely allow users to communicate nuanced feedback to the system in real time, resulting in disengagement or overstimulation.

While various smart devices and haptic systems have emerged for enhancing sexual experience, these are often standalone, lacking integration with immersive environments. Current systems do not offer a unified architecture in which VR environments, biofeedback, user gestures, and wearable devices operate in real-time synchrony. Nor do they embed therapeutic safeguards, such as content modulation based on emotional state, consent re-confirmation, or scenario adaptation according to stress signals.

Accordingly, there remains a need for a unified platform that delivers immersive, emotionally adaptive VR experiences specifically designed for sexual wellness. Such a system must support controller-free interaction, scenario-specific logic for smart device management, and real-time content adaptation based on behavioral and physiological input. It should allow users to explore intimacy, rebuild trust, or engage in fantasy within a safe, hands-free, and customizable virtual environment—filling a gap left by entertainment VR, standalone adult devices, and conventional therapy tools.

### 3. SUMMARY OF THE INVENTION

The present invention relates to a virtual reality (VR) system and method for delivering immersive intimate experiences in support of sexual wellness. The system comprises a controller-free VR headset configured for upper-body or full-body tracking, one or more wearable smart stimulation devices operably synchronized with virtual scenarios, and a simulation engine capable of real-time adaptation based on user input and contextual feedback.

The system operates through a dedicated launcher platform, which enables users to browse, download, and manage immersive simulations. Each simulation contains its own interaction logic for controlling synchronized smart devices and interpreting user input, allowing for scenario-specific feedback, pacing, and safety mechanisms. This modular structure allows individual simulations to manage device behavior and emotional responsiveness according to therapeutic or experiential goals.

Wearable smart devices are used to deliver hands-free stimulation, including haptic, thermal, vibratory, or pressure-based feedback. These devices may incorporate embedded sensors such as inertial measurement units (IMUs), which can detect motion, pressure, or localized physiological changes. Sensor data is used by the simulation engine to trigger content adjustments, synchronize feedback, or initiate safety responses based on user comfort or emotional state.

The system supports real-time user interaction through gaze tracking, hand or arm movement detection, and voice commands, eliminating the need for handheld controllers. Adaptive features may include automatic scenario pausing, intensity reduction, or grounding prompts when stress markers or behavioral cues exceed user-calibrated thresholds.

The invention further includes a launcher interface that allows users to browse, download, and initiate VR simulations. Calibration, device pairing, and settings management are handled within each individual simulation, enabling context-aware configuration and ensuring tight integration between content and hardware. The platform is designed to support ongoing expansion through additional smart devices, scenario modules, and third-party integrations.

By combining immersive simulation, synchronized stimulation devices, real-time adaptive logic, and modular software deployment, the invention delivers a personalized, emotionally attuned VR platform for sexual wellness, intimacy rehabilitation, and therapeutic self-exploration.

### 4. BRIEF DESCRIPTION OF VIEWS OF THE DRAWING

FIG. 1 is a schematic diagram illustrating a system overview of a virtual reality (VR) platform for immersive simulation experiences, showing the interaction between a VR headset, wearable smart stimulation devices, a simulation engine, and a launcher interface, according to one embodiment of the present invention.

FIG. 2 is a schematic diagram illustrating a user experience flow for an immersive interaction system, according to one embodiment of the present invention. The figure depicts how user-initiated inputs, including both movement and voice commands, are detected and interpreted by the simulation, triggering adaptive responses and generating synchronized device feedback. This creates a continuous, real-time loop of interaction between the user and the system.

FIG. 3 is a schematic diagram illustrating a sensor and motion feedback loop for immersive interaction, according to one embodiment of the present invention. The figure shows how user-initiated inputs are detected by system sensors, processed by adaptive simulation logic, and used to trigger real-time updates and physical device feedback, forming a closed loop of continuous user engagement.

FIG. 4 is a schematic diagram illustrating a simplified logic feedback loop for immersive user interaction, according to one embodiment of the present invention. The figure demonstrates how input from sensors (such as IMUs, gaze tracking, hand tracking, etc.) is processed by the simulation engine, which then generates corresponding output controls through connected smart devices. The sensory feedback from the output controls is fed back into the input sensors, creating a continuous loop of real-time adaptation and interaction to enhance the user experience.

### 5. DETAILED DESCRIPTION OF THE INVENTION

The present invention provides a comprehensive Virtual Reality (VR) platform for immersive simulation experiences in the field of sexual wellness. This system addresses the need for adaptive, therapeutic, and interactive engagement by integrating real-time biofeedback, a responsive simulation engine, and synchronized smart devices. The invention offers a safe, personalized, and immersive experience for users.

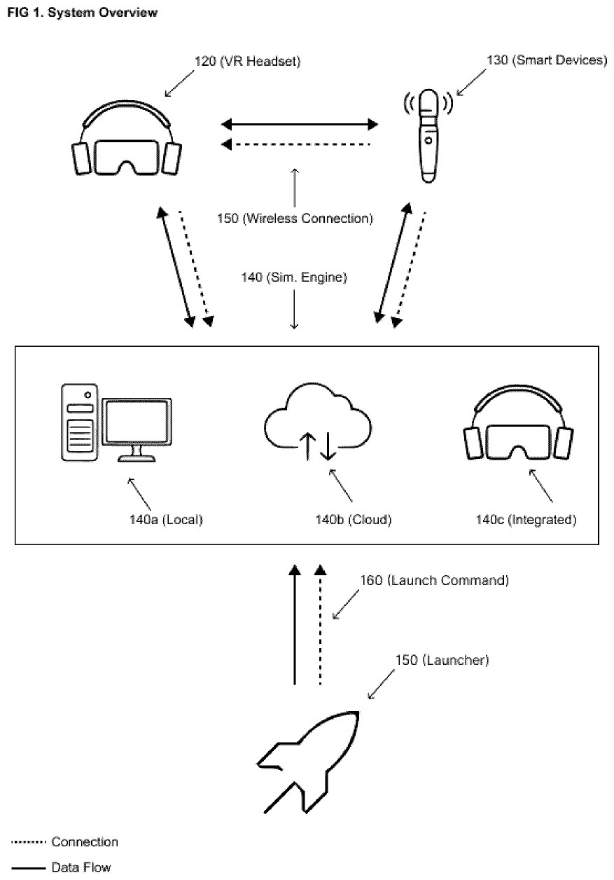

### System Overview (FIG. 1)

As shown in FIG. 1, the system comprises four primary components.

A VR headset (120) delivers immersive audiovisual content and tracks user motion through inertial measurement units (IMUs), gaze tracking, motion tracking including hand, arm, or full-body tracking, and voice recognition. The VR headset communicates bidirectionally with the simulation engine and smart devices.

Smart stimulation devices (130) include haptic actuators and wearable stimulatory technologies. These devices are wirelessly synchronized with the VR headset to deliver tactile feedback that enhances immersion, wherein the feedback adapts to user input and physiological data.

A simulation engine (140) processes user input, adapts the simulation in real time, and manages the overall experience. The simulation engine may be implemented in one of the following configurations:
(a) a local configuration (140a) comprising processing on a dedicated local unit;
(b) a cloud-based configuration (140b) enabling remote processing and dynamic content updates; or
(c) an integrated configuration (140c) embedded within the VR headset for portability.

Wireless protocols including Bluetooth and Wi-Fi ensure real-time synchronization among system components.

A launcher platform (150) serves as a user interface for selecting and launching simulations. The launcher platform sends launch commands (160) to the simulation engine, which then manages all device connections and session processes.

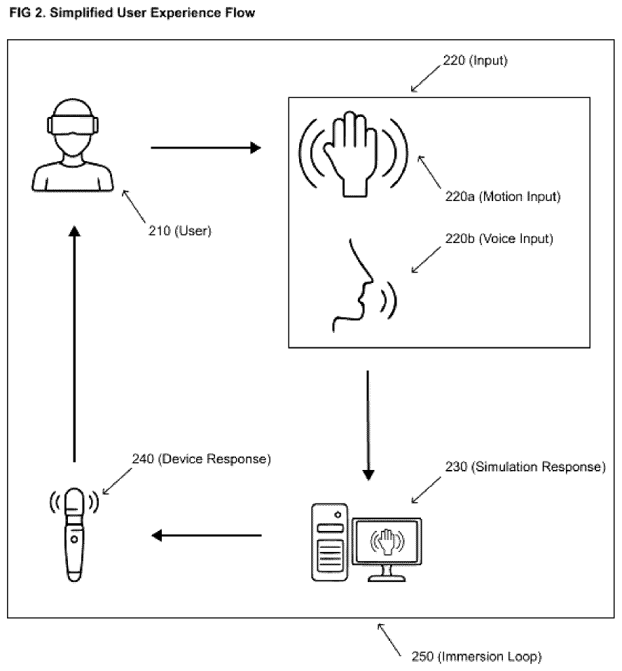

### Immersion Flow and User Interaction (FIG. 2)

Upon launch, the user dons the VR headset, initiating a continuous feedback loop. User inputs (220)—including movements (220a) and voice commands (220b)—are captured by the headset and transmitted to the simulation engine (140). The engine processes these inputs to dynamically update the simulation (230), adapting visual, auditory, and interactive elements.

Synchronized feedback is then delivered to the user through the VR environment and smart devices. These responses reinforce immersion and emotional engagement. Smart stimulation devices (130) respond based on the simulation's logic, creating physical sensations aligned with the virtual experience.

This creates a real-time immersion loop (250), where user actions continuously influence the simulation and elicit responsive feedback.

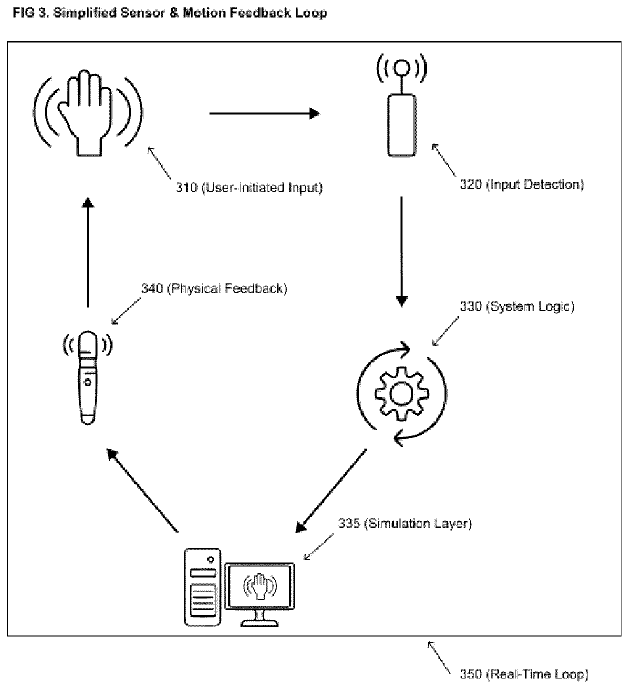

### Closed Feedback Loop (FIG. 3 & 4)

User input (310)—gestures, posture, voice, or physiological signals—is detected by sensors (320) embedded in the headset and smart devices. These may include IMUs, optical trackers, microphones, or biometric sensors. The simulation engine (330) interprets the data to determine context-specific responses.

Based on the analysis, the simulation layer (335) is updated to reflect emotional and behavioral context—modifying pacing, visuals, or narrative flow. Simultaneously, smart devices deliver physical feedback (340) such as haptic or thermal sensations.

The loop continues (350/440) as the system monitors the user's reactions, updating the simulation and device outputs accordingly. This ensures the experience remains adaptive and personalized throughout.

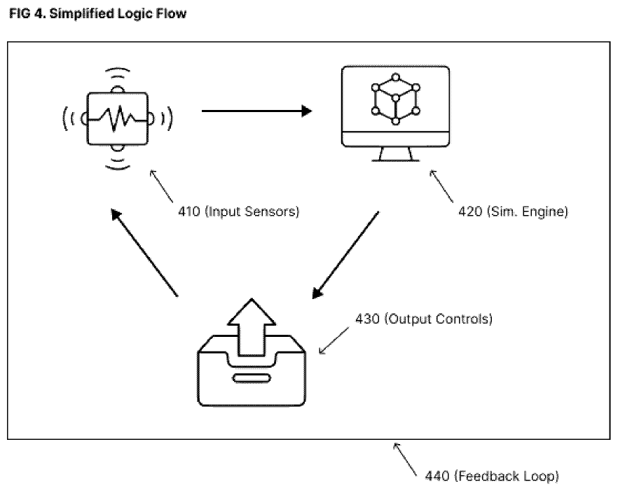

### System Logic Integration (FIG. 4)

Input sensors (410) capture user behavior and physiological states. The simulation engine (420) processes this data and outputs control signals to smart devices (430), which are synchronized with simulation content. The system maintains a closed loop (440), continuously adapting to the user's needs.

This architecture allows for immersive, emotionally safe, and therapeutically relevant simulations, reinforcing the user's presence and engagement through a seamless blend of visual, auditory, and physical feedback.

The present invention offers significant improvements over existing VR-based interactive systems and sexual wellness devices, by integrating immersive, real-time interaction between the user, VR content, and synchronized smart stimulation devices. Unlike prior art, which typically focuses on either visual/auditory immersion or tactile feedback in isolation, the present system uniquely combines dynamic, context-aware simulation with real-time biofeedback to deliver a personalized, adaptive experience.

Real-Time Feedback Integration: Existing systems often rely on pre-programmed responses based on user input or predetermined environmental triggers. In contrast, the present invention continuously adapts the experience based on real-time physiological and behavioral data, such as heart rate, motion, and gaze patterns, captured by sensors within the VR headset and external devices. The system uses this data to adjust both visual content and smart device feedback (e.g., tactile, thermal, or mechanical sensations), ensuring that the immersive experience remains in sync with the user's physical and emotional state.

Middleware Simulation Engine: A novel aspect of the invention is the role of the simulation engine as a centralized processing module that acts as middleware between the user's sensor inputs and the output controls (smart devices). Prior solutions typically rely on disconnected feedback mechanisms or do not dynamically adjust based on continuous input data. The invention's middleware logic ensures seamless interaction by interpreting sensor data in real-time and triggering synchronized changes to both the VR environment and smart stimulation devices, thus enhancing user engagement and immersion.

Closed Feedback Loop for Enhanced Personalization: The system implements a continuous closed feedback loop, where user reactions to stimuli directly inform further adjustments in the virtual environment and device responses. This loop creates a dynamic experience that adapts to user preferences, ensuring comfort, safety, and engagement. Unlike prior art, which lacks this real-time adaptation mechanism, the invention's closed-loop ensures that the experience evolves with the user, allowing for personalized interaction that can respond to both physical and emotional shifts in real-time.

Multi-Device Synchronization: The invention uniquely integrates a variety of smart stimulation devices with the VR headset, ensuring that all components are fully synchronized during operation. While previous systems may rely on isolated devices or lack the necessary coordination between the virtual and physical components, the present system ensures harmonized control over all devices, optimizing the user experience through real-time interaction and synchronized feedback.

These distinguishing features make the present invention a novel and more effective solution compared to existing technologies in the field of immersive VR experiences and sexual wellness, offering enhanced user engagement, adaptability, and immersion.

The preferred embodiment of the present invention consists of several key hardware and software components that work together seamlessly to deliver an immersive, adaptive virtual reality (VR) experience.

The system uses a VR headset (120) that incorporates eye tracking and/or hand tracking to capture user inputs, allowing for intuitive interaction with the virtual environment. As immersive action sensing (IAS) technologies evolve, the system is designed to integrate additional tracking capabilities, such as arm tracking, leg tracking, and potentially full-body tracking.

The system also incorporates smart stimulation devices (130), which are essential for delivering immersive sensory experiences to the user. They are linked to the VR environment and adjust their output based on the user's interactions, creating a direct sensory connection between the user and the simulation.

To further enhance the realism of these interactions, the smart stimulation devices are equipped with Integrated Measurement Units (IMUs). These IMUs help track the user's movements, posture, and physiological responses, enabling the system to dynamically adapt the feedback according to the user's actions and physical state. By integrating IMUs, the system offers more nuanced and responsive tactile feedback, enriching the user's sense of immersion and reinforcing the connection between the user and the virtual world.

At the current stage, a PC or similar computing device is required to handle the computational processing of the simulation, as it provides sufficient power to support real-time data processing, system synchronization, and content generation. As technology advances, the invention is designed to evolve toward all-in-one VR headsets capable of handling both processing and content delivery, or even cloud-based processing, to further enhance portability and scalability.

The software side of the system includes the launcher, simulation engine and device synchronization. The simulation engine is responsible for synchronizing the smart stimulation devices with the VR content.

As the system evolves, future implementations may incorporate artificial intelligence (AI) to further enhance user interaction and personalization. AI integration could enable the simulation engine to learn from user behavior over time, optimizing content pacing, adjusting feedback sensitivity, and predicting user preferences for a more intuitive and emotionally responsive experience. This would allow the system to deliver increasingly tailored simulations that adapt not only in real-time but also based on learned patterns and evolving user needs.

The best mode of carrying out the invention combines state-of-the-art VR hardware, adaptive simulation software, and synchronized real-time feedback mechanisms. The system is designed to evolve alongside technological advancements, ensuring that users experience increasingly personalized and immersive VR simulations. It maintains flexibility for future upgrades, including the adoption of all-in-one VR headsets, cloud-based processing, and artificial intelligence (AI) integration.

The following examples illustrate specific embodiments of the invention and are intended to demonstrate practical applications of the system in various use cases. Each scenario utilizes the core components of the invention—including the VR headset, simulation engine, and smart stimulation devices—to deliver an adaptive, immersive experience tailored to the user's needs and preferences.

### Example Scenario 1: Trauma Recovery—Pleasure-Focused Intimacy Rebuilding

In this embodiment, the system is configured to support a female user in rebuilding comfort with receiving pleasure in a non-threatening, emotionally supportive setting. Upon launching the simulation via the launcher, the user selects a trauma recovery scenario. The simulation engine initiates a serene virtual environment—such as a softly lit forest glade or a private indoor retreat—with ambient sound and calming visuals.

The session begins with guided grounding exercises, such as breathwork and eye-gaze calibration, to establish a sense of presence and emotional safety. The user is gradually introduced to a calm, non-verbal avatar whose actions—such as gentle caressing or holding—are triggered only upon explicit user consent, given through verbal commands or subtle gestures (e.g., hand movement, head nodding).

Smart stimulation devices synchronized with the avatar's actions deliver soft tactile feedback, such as warmth or gentle pressure. Integrated IMU sensors monitor the user's physiological cues (e.g., stillness, gaze aversion, micro-movements), which are processed by the simulation engine to adjust pacing, avatar proximity, and interaction intensity in real time.

The session may include affirming audio or soft verbal scripting to reinforce the user's agency and comfort. No penetrative or overtly sexual actions are presented. The purpose of this scenario is to facilitate reconnection with pleasurable sensations in a controlled, safe, and responsive manner.

### Example Scenario 2: Fantasy Exploration—Roleplay-Led Immersion for Men

In this embodiment, a male user engages with a scenario focused on fantasy roleplay within a controlled, consent-driven framework. Upon initiating the simulation, the user is placed in a semi-scripted environment featuring a responsive avatar and a narrative arc supported by voiceovers and prompts.

The user adopts an active role and may initiate actions through gesture or voice input. Smart haptic devices respond to both user behavior and avatar interaction, delivering synchronized tactile feedback.

The system continuously monitors physiological signals via IMUs embedded in wearable devices. If the system detects elevated arousal or stress (e.g., rapid motion followed by sudden stillness or pressure spikes), the simulation transitions into a resolution phase. This may include slowing down the pacing, softening lighting or audio, and guiding the user through grounding exercises.

This embodiment allows the user to safely explore dominant or expressive roles, with continuous emotional and physical calibration to ensure well-being and agency throughout the session.

### Example Scenario 3: Movement Assistance—Guided Engagement With Command-Based Control

In this embodiment, a user with limited lower-body mobility initiates a session designed for low-effort yet intimate interaction. During calibration, the system identifies mobility constraints and adjusts control schemes to prioritize eye tracking, hand gestures, and voice input.

The simulation introduces a responsive avatar who assumes an active role in the interaction, guided by the user's directional cues. For instance, the avatar may ask, “Would you like me to come closer? ”with the user replying via nod, voice, or gesture.

The simulation engine interprets these inputs to control scene transitions, avatar pacing, and device stimulation levels. Smart haptic devices, worn or positioned according to the user's needs, deliver tactile feedback that corresponds with the avatar's movements, creating a sense of embodied presence without requiring full-body motion.

Physiological monitoring ensures that the session remains within safe parameters. If the system detects prolonged stillness, stress markers, or non-responsiveness, the avatar adjusts tone, offers reassurance, or prompts for confirmation to continue or end the session. This embodiment supports dignified and immersive engagement, adapted to the user's physical capacity.

1. A system for delivering immersive sexual wellness experiences, comprising:
a virtual reality (VR) headset comprising at least an eye tracking module and a hand tracking module configured to capture user behavior;
one or more smart stimulation devices configured to deliver physical feedback to a user, the feedback comprising at least one of: tactile, thermal, or
mechanical output;
a simulation engine operatively coupled to the VR headset and the stimulation devices, the simulation engine configured to:
receive behavioral data from the VR headset and sensor data from the stimulation devices;
adapt a virtual environment in real time based on the received data; and
synchronize operation of the stimulation devices with events rendered in the virtual environment;
a launcher module configured to initialize simulations, perform hardware readiness checks, and issue a launch command to the simulation engine;
a wireless communication interface configured to maintain low-latency data exchange between the VR headset, the simulation engine, and the stimulation devices;
wherein the system includes a closed feedback loop that continuously updates the simulation and device outputs based on ongoing user interaction and input data.

2. The system of claim 1, wherein the simulation engine is hosted on at least one of:
a local computing device;
a cloud-based computing platform; or
a processing unit integrated within the VR headset.

3. The system of claim 1, wherein each smart stimulation device comprises at least one inertial measurement unit (IMU) for tracking user movement, orientation, or physical feedback.

4. The system of claim 1, wherein the simulation engine detects user physiological responses selected from the group consisting of: gaze aversion, heart rate variability, posture changes, stillness, and motion patterns.

5. The system of claim 1, wherein the simulation engine selects and adapts simulation content from a set of pre-configured scenarios including:
a trauma recovery simulation involving emotionally supportive interaction;
a fantasy exploration simulation involving user-driven roleplay;
a movement assistance simulation involving guided engagement with limited input.

6. The system of claim 1, wherein each scenario includes an avatar character whose actions are modulated by the user's behavior, physiological responses, or verbal/gesture input.

7. The system of claim 1, further comprising a calibration module configured to:
detect baseline physical capability and user preferences; and
adjust device parameters, avatar behavior, and environmental settings accordingly.

8. The system of claim 1, wherein the simulation engine includes an artificial intelligence module trained to:
personalize simulations based on historical user behavior;
recommend new simulation content;
optimize device timing and intensity patterns based on prior session data.

9. The system of claim 1, wherein system components communicate using one of:
Bluetooth, Wi-Fi, or a proprietary low-latency wireless protocol.

10. The system of claim 1, wherein device synchronization is performed within the simulation engine logic embedded in the simulation software.

11. A method of delivering an adaptive immersive experience using a virtual reality-based sexual wellness system, comprising:
initializing a simulation via a launcher module;
calibrating one or more smart stimulation devices and a VR headset based on
user input and physical parameters;
detecting user behavior via eye tracking and hand tracking sensors;
delivering a virtual simulation scene through the VR headset;
synchronizing haptic output from the stimulation devices with events occurring in the simulation;
monitoring user responses and dynamically adjusting simulation pacing, avatar behavior, and device feedback;
optionally using artificial intelligence to refine user preferences and update future simulation behavior.
